(19) United States
(12) Patent Application Publication
(10) Pub. No.: US 2005/0159780 A1
(43) Pub. Date: Jul. 21, 2005

(54) Navigated heat treatment of tumors with enzyme-coated iron particles

(76) Inventor: Norbert Rahn, Forchheim (DE)

Correspondence Address: SCHIFF HARDIN, LLP;PATENT DEPARTMENT, 6600 SEARS TOWER, CHICAGO, IL, 60606-6473, US

(21) Appl. No.: 11/015,164

(22) Filed: Dec. 17, 2004

(30) Foreign Application Priority Data
Dec. 17, 2003 (DE) 103 59 252.0

(51) Int. Cl. A61B 5/055; A61K 38/43

(52) U.S. Cl. 607/1; 424/94.1

(57) ABSTRACT

An apparatus for heat treatment of tumors has an injection device for introduction of enzyme-coated iron particles into the tumor as well as a magnet system disposed outside of the patient for the generation of an alternating magnetic field for heating the iron particles and a navigation system with at least one position/orientation sensor disposed on the injection device.

### BACKGROUND OF THE INVENTION

1. Field of the Invention

The present invention concerns an apparatus for heat treatment of tumors, of the type having with an injection device for introduction of enzyme-coated iron particles into the tumor.

2. Description of the Prior Art

A newly developed tumor therapy has been described in recent medical journals, in which therapy tumors in neurons (for example glioblastomas) or in the prostate region or mammary region are treated by means of magnetic fluid hyperthermy. The therapy method, in which miniscule iron particles are directly injected into the tumors and then the malignant cells are destroyed by subsequent overheating by external alternating magnetic fields, enables a targeted overheating of only the region of the tumor, for example to 45° C. In this treatment, the patients generally are subsequently irradiated as well in order to also reach the few cancer cells still surviving

The iron particles are coated with a molecular layer that has an affinity to new cell membranes. Due to the high division frequency of tumor cells, there are many of these new membranes in tumors. As a consequence of the enzyme coating, the iron particles adhere to these and are transferred into the cells. There can be millions of iron particles per cell.

In order to achieve such concentrations, the particles are injected directly into the tumor via a small-bore hole, for example in the roof of the skill. Tumors that lie at sensitive locations such as the speech center can also be reached stereotactically.

After the particles are excited by an externally generated alternating magnetic field and thereby become heated and emit heat to the tumor cells, the temperature can be monitored via a thin temperature probe that is likewise pushed into the tumor through the borehole. The particles heat the tumor, damage the cells and make them sensitive for the subsequently applied irradiation.

For various tumor types, specific molecular coatings of the iron particles (fashioned for the most part as nanoparticles) are used. Specific coatings for further tumors such a prostate carcinomas are also commercially available. An alternating magnetic field applicator MFH 300 F suitable for the treatment is provided by the firm MFH Hyperthermiesysteme.

This minimally invasive method, which could be an excellent alternative to operation in the case of small mammary carcinomas, succeeds or fails dependent the precision of the introduction of the iron particles into the tumor.

### SUMMARY OF THE INVENTION

An object of the present invention is to provide an apparatus of the above-cited type that allows, in a simple manner, a controlled introduction of the iron particles into the tumor to be achieved.

This object is achieved in accordance with the invention by an apparatus of the type initially described that is equipped with a navigation system employing at least one position/orientation sensor on the injection device. The apparatus preferably has a screen for visualization of a 3D image data set of the tumor region as well as to mix the image of the distal end of the injection device into the 3D image.

By the inventive use of a known navigation system for precise guidance of the injection device with the aid of position/orientation sensors on the injection device, a very gentle, exact introduction of the iron particles into the tumor can be achieved even in the case of tumors that are buried very deep, in particular when a flexible injection device is used that does not have to simply pierce the tissue lying between body surface and the tumor, but also can move around individual organs.

Alternatively to the use of an optical navigation system with a position/orientation sensor at the distal end of a rigid injection device, naturally an electromagnetic navigation system can be used in which the position/orientation sensor is then preferably disposed at the proximal end of the injection device, thus precisely at the location whose position should be monitored (with regard to its movement) in the targeted injection of the iron particles into the tumor, in order to precisely guide the deposit of the nanoparticles from the injection examination volume at the desired location in connection with the mixing of the 3D image of the injection region.

In an embodiment of the invention, one or more reference sensors that enable a computerized elimination and compensation of patient movements are arranged on the patient surface.

In order to achieve a targeted introduction of the iron particles into the entire tumor tissue in the case of non-spherical, somewhat elongated tumors, as well as into tumors that are deformed and reticulated in different directions, in an embodiment of the invention the magnet system is fashioned for generation of an additional variable but constant (static; d.c.) field for movement of the iron particles in the desired directions. Permanent magnets or electromagnets that draw the iron particles in defined, predeterminable directions serve for this purpose. So that this movement of the iron particles is exactly executed via a controllable external magnetic field, it is necessary to provide a device for density determination of the body tissue in the injection region. Such a device in the simplest case can be a measurement evaluation device for evaluation of CT exposures, since the attenuation of the x-rays in such exposures gives a very good representation of the tissue density.

When the spatially dependent density of the tumor tissue has first been determined in this manner, the time-dependent and spatially dependent alignment of the external constant magnetic field is calculated using simulation models or algorithms such that the desired distribution of the metal particles is achieved. This spatially- and time-dependent magnetic field is applied according to the simulation, so the desired distribution of the metal particles occurs.

### DESCRIPTION OF THE DRAWINGS

FIG. 1 is a schematic representation of an inventive apparatus for navigation of an injection device with navigation sensor at the distal end of the injection device.

FIG. 2 is a schematic representation of another embodiment of the inventive apparatus, with an electromagnetic navigation sensor at the proximal end of the injection device.

FIG. 3 schematically illustrates, corresponding with FIGS. 1 and 2, an optical navigation system with a distal position/orientation sensor on the injection device.

### DESCRIPTION OF THE PREFERRED EMBODIMENTS

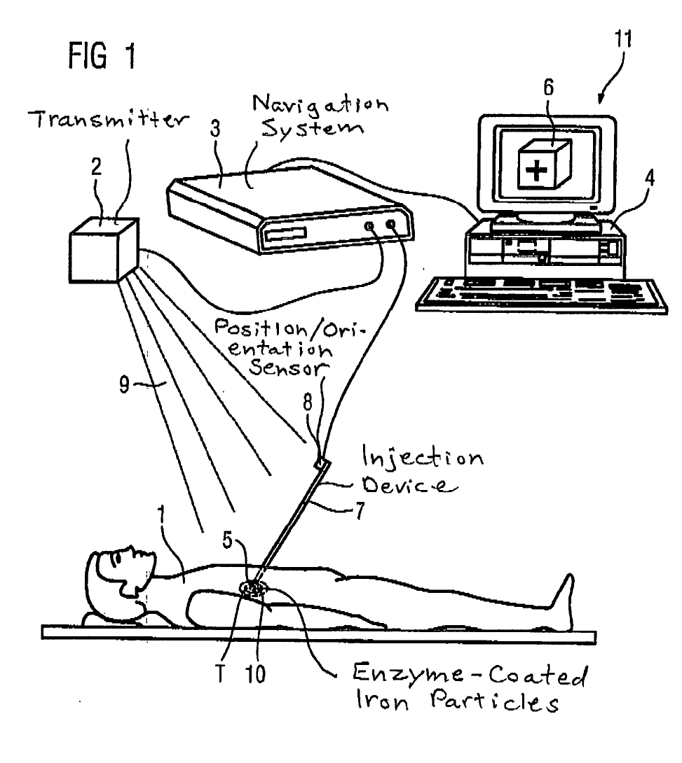

In FIGS. 1, 2 and 3, an injection device, for example a rigid needle, is be controllably introduced into a patient 1 using a navigation system 3 having a transmitter 2 and a navigation computer 4. The navigation proceeds such that the distal end 5 of the injection device 7 can be precisely introduced into a tumor T located in the body of the patient 1.

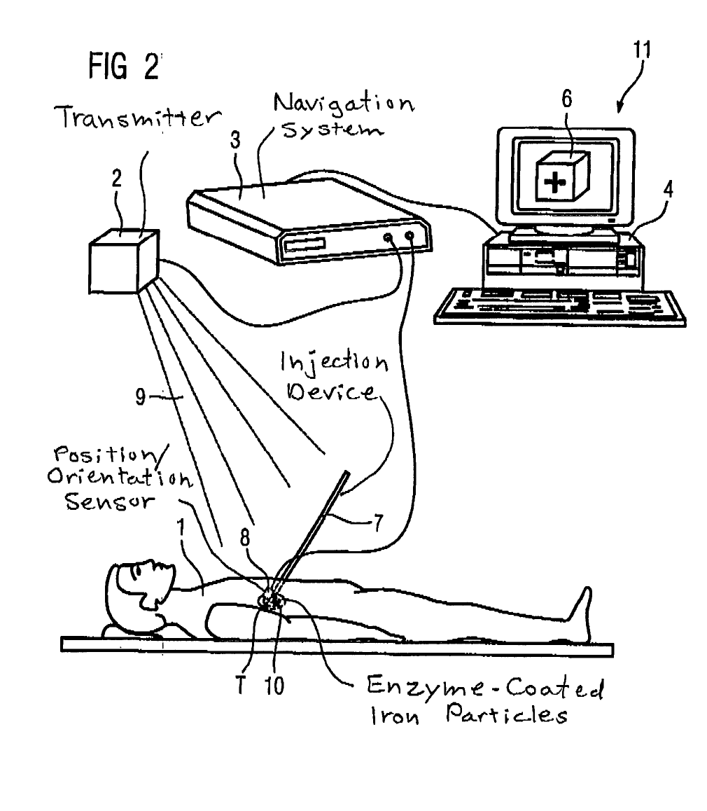

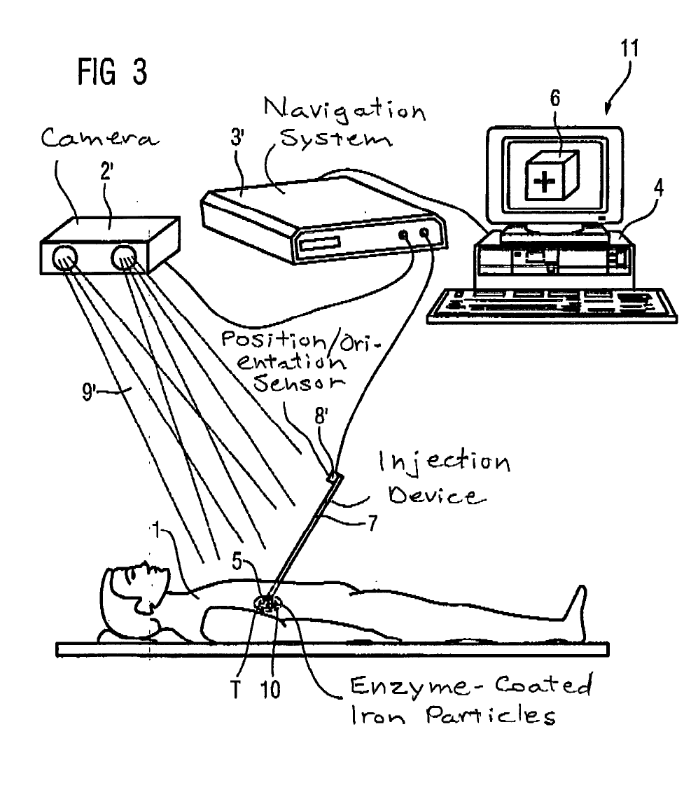

For this purpose, one position/orientation sensor 8 is provided that can be arranged either at the distal end 5 (see FIG. 1) or at the proximal end (FIGS. 2, 3 of the injection device 7). In addition to a position sensor that only indicates the three spatial coordinates, an orientation sensor can also be used that additionally detects the three spatial angles, such that via the orientation sensor not only the position of the sensor but rather also the direction in which it moves can be detected. This is important when the sensor is arranged at the distal end of the injection device 7, thus outside of the body.

The injection device 7, as an alternative to a rigid needle, could also be a mobile injection device. In the latter case only the use of an arrangement similar to that in FIG. 2 is considered, in which the position/orientation sensor is at the proximal end (thus in the internal end) of the injection device 7. The position/orientation sensor is again designated at 8 and the electromagnetic field of the navigation system is designated with 9.

The magnet system surrounding the patient for generation of an alternating magnetic field is not also shown, for clarity.

The arrangement according to FIG. 2 differs from that according to FIG. 1 only in that the electromagnetic position/orientation sensor 8 is arranged at the proximal (internal) end of the injection device 7.

The arrangement according to FIG. 3 shows a device with an optical navigation system 3′, wherein 2′ is the camera of this optical navigation system that corresponds to the transmitter 2 of the electromagnetic navigation system 3 according to FIGS. 1 and 2. In such an optical navigation system 3′, the orientation sensor 8′ (in this case an orientation sensor is needed to detect 6 degrees of freedom, in contrast to the proximal arrangement of a simple position sensor) can only be arranged at the distal end of the injection device 7, which in turn has the consequence that the injection device 7 must be rigid, for example as a needle. The optical field of view of the navigation system 3′ is designated at 9′ and the enzyme-coated iron particles that should be precisely introduced into the tumor T with the aid of the injection device 7 are again indicated with 10, as in FIGS. 1 and 2.

A representation of the current position of the proximal end 5 of the injection device 7 is mixed into the 3D image data set 6 (visualized on the screen 11) of the injection area, thus the tumor T, such that with the aid of this mixing a very simple, exact navigation of the proximal end of the injection device 7 into the tumor T can ensue.

As was the case for the magnetic field for the generation of the alternating magnetic field to heat the iron particles, the magnetic field for the generation of an additional variable constant field for movement of the iron particles in the desired directions, and thus for optimal distribution of these iron particles in an unusually shaped tumor, has not been included in the drawings, for clarity.

Although modifications and changes may be suggested by those skilled in the art, it is the intention of the inventor to embody within the patent warranted hereon all changes and modifications as reasonably and properly come within the scope of his contribution to the art.

1. An apparatus for heat treatment of tumors, comprising:
an injection device adapted for in vivo insertion into a tumor for introducing enzyme-coated iron particles into the tumors;
a magnet system disposed outside of the patient for generating an alternating magnetic field for heating the iron particles in the tumor; and
a navigation system for assisting in guiding introduction of said injection device, said navigation system including at least one position/orientation sensor disposed on said injection device.

2. An apparatus as claimed in claim 1 wherein said injection device is a rigid injection device having a distal end adapted for insertion into said tumor, and an opposite proximal end, and wherein said navigation system is an optical navigation system with said position/orientation sensor disposed at said distal end of said injection device.

3. An apparatus as claimed in claim 1 wherein said injection device has a distal end adapted for introduction into said tumor, and an opposite proximal end, and wherein said navigation system is an electromagnetic navigation system having said position/orientation sensor disposed at said proximal end of said injection device.

4. An apparatus as claimed in claim 1 wherein said injection device has a distal end adapted for introduction into the tumor, and an opposite proximal end, and wherein said apparatus comprises a display screen connected to said navigation system for displaying a 3D image dataset of said tumor, and for mixing a representation of said proximal end of said injection device into the displayed 3D image dataset of the tumor.

5. An apparatus as claimed in claim 1 comprising a computer connected to said display screen and to said navigation system, and wherein said apparatus comprises a reference sensor adapted to be disposed on a surface of a patient in whom the tumor is disposed, said reference sensor supplying a signal to the computer and said computer making computations dependent on said signal from said reference sensor to eliminate patient movements from affecting said 3D image dataset of said tumor with said representation of said proximal end of said injection device mixed therein.

6. An apparatus as claimed in claim 1 comprising a magnet system for generating a selectable, constant magnetic field for controlling movement of said iron particles in selected directions.

7. An apparatus as claimed in claim 1 comprising a device for making a density determination of body tissue in a region of a patient containing said tumor.
